(19) United States
(12) Patent Application Publication
(10) Pub. No.: US 2020/0118655 A1
(43) Pub. Date: Apr. 16, 2020

(54) DYNAMIC CONTEXTUAL INTERFACES TO ELECTRONIC MEDICAL RECORDS DATABASE

(71) Applicants: Natalie Hoad-Reddick, Meaford (CA); Wade Hatler, RENTON, WA (US)

(72) Inventors: Natalie Hoad-Reddick, Meaford (CA); Wade Hatler, RENTON, WA (US)

(21) Appl. No.: 16/653,037

(22) Filed: Oct. 15, 2019

Related U.S. Application Data
(60) Provisional application No. 62/745,740, filed on Oct. 15, 2018.
(60) Provisional application No. 62/773,633, filed on Nov. 30, 2018.

(51) Int. Cl. G16H 10/60; G06Q 10/10; G16H 50/70; G06K 9/62

(52) U.S. Cl. CPC G16H 10/60; G06K 9/6267; G16H 50/70; G06Q 10/10

(57) ABSTRACT

A system and method for presenting a dynamic patient whiteboard may include displaying a custom user interface including the dynamic patient whiteboard. The custom user interface may display a patient list including a plurality of patients and related disease state, cancer type, or treatment protocol, in an example, The dynamic patient whiteboard may include a plurality of oncology-related tasks, and each oncology-related task of the plurality of oncology-related tasks may include a task status. An electronic medical records (EMR) database may be accessed. A processor may be used to determine whether a task status for an oncology-related task has been updated, and in response optionally update the dynamic patient whiteboard to reflect the updated task status. Context information may be displayed for the updated task or other tasks, for example based on a particular user identity.

### CLAIM OF PRIORITY

This application claims the benefit of U.S. Provisional Patent Application Ser. No. 62/745,740, filed on Oct. 15, 2018 and U.S. Provisional Patent Application Ser. No. 62/773,633, filed on Nov. 30, 2018, the benefit of priority of which are claimed hereby, and which are incorporated by reference herein in their entirety.

### TECHNICAL FIELD

Embodiments of the present disclosure pertain generally to dynamic interfaces to an electronic medical records (EMR) database. In particular, the present disclosure pertains to contextual data displays tailored to specific users, dynamic electronic whiteboards, and automated data capture utilizing voice recognition capabilities.

### BACKGROUND

Currently, most medical treatments are designed for the average patient. As a result of this “one-size-fits-all-approach,” treatments can be very successful for some patients but not for others. For, one size does not fit everyone. But not everything is so generic. For instance, if you need glasses, you are given a prescription specific to adjust your eyes; if you have an allergy, the tests determine exactly what you are allergic to; and if you need a blood transfusion, it must match your blood exactly.

This is changing with the emergence of precision medicine, an innovative approach to disease prevention and treatment that considers individual differences. For instance, variation in people's genes, environments, lifestyles and even in the microscopic organisms that are living inside of us. Many diverse sources of data are used in precision medicine, including medical records; profiles of the patient's genes, metabolites (chemical makeup), and microorganisms in and on the body; environmental and lifestyle data; patient-generated information; and personal device and sensor data. And understanding individual variations in the enzymes that process drugs can also help a doctor prescribe the right dose of the right medication.

Understanding how genetic variations contribute to health is just one aspect of precision medicine. While the genome is set for life, the expression of our genes fluctuates over time and in response to the environment. Additional approaches to precision medicine involve measuring levels of proteins, RNAs, or metabolic products. Along with genomics, proteomics, epigenomics, and metabolomics can help inform medical choices for individual patients.

Precision medicine gives clinicians tools to better understand the complex mechanisms underlying a patient's health, disease, or condition, and to better predict which treatments will be most effective.

For example, in the traditional approach to cancer treatment, doctors decide whether to prescribe chemotherapy based on the stage of an individual's cancer. People with early-stage cancers are usually not given chemotherapy, which typically costs many tens of thousands of dollars and often causes unpleasant side effects. Yet some early-stage cancers progress rapidly, and those individuals eventually end up getting chemotherapy when their cancer is at a more-advanced stage.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an example user interface for accessing different aspects of the systems and methods described herein, in accordance with at least one example of this disclosure.

FIGS. 2A-2B illustrate a dynamic patient whiteboard in a custom user interface, in accordance with at least one example of this disclosure.

FIG. 3 illustrates a smart form in a custom user interface, in accordance with at least one example of this disclosure.

FIG. 4 illustrates a reverse electronic medical records (EMR) workflow, in accordance with at least one example of this disclosure.

FIGS. 5A-5B, 6A-6H, 7A-7E, and 8 illustrate example user interface components displaying aspects of a reverse EMR system, in accordance with at least one example of this disclosure.

FIG. 9 illustrates a block diagram of a system for presenting a dynamic patient whiteboard, in accordance with at least one example of this disclosure.

FIG. 10 illustrates a flowchart showing a technique for presenting a dynamic patient whiteboard, in accordance with at least one example of this disclosure.

### DETAILED DESCRIPTION

Precision medicine allows treatments to be tailored to specific characteristics of individuals, such as a person's genetic makeup, or the genetic profile of an individual's tumor. This is leading to a transformation in the way we can treat diseases such as cancer. Patients with breast, lung, and colorectal cancers, as well as melanomas and leukemias, for instance, routinely undergo molecular testing as part of patient care, enabling physicians to select treatments that improve chances of survival and reduce exposure to adverse effects.

For example, even if someone inherits genetic variations that make them susceptible to skin cancer, they can decrease their chances of getting cancer by protecting themselves from the sun. Precision medicine involves understanding how factors from the environment interact with genetic variations to influence health. When combined with information about a person's environment, genetic information becomes even more powerful.

Another example is of precision medicine test designed to evaluate the aggressiveness of lung cancer. A genetic test was given to people who were newly diagnosed and whose cancer was still at an early stage. When the test indicated that a person had an aggressive form of cancer, they were offered chemotherapy, e.g., most effective at treating cancers that are growing and dividing rapidly. However, when the test indicated that a person had a less-aggressive cancer, precision medicine allowed for this patient not to be offered chemotherapy. This spared the patient the cost and the side effects of a treatment that was less likely to be effective for them at this early stage.

Further, precision medicine may combine the latest in stem cell science with 3d printing technology to build replacement skin, blood vessels, and bones. It includes techniques for engineering a patient's own immune cells to attack cancer and other diseases, and computer algorithms for building customized diets for diabetic patients. Precision medicine also includes approaches to diagnostics, prevention, and screening, for example, methods for identifying those who are at risk before disease strikes, analytical tools for predicting which prevention strategies will work best for which patients, screening methods that can identify early signs of disease before symptoms emerge, diagnostic methods for identifying subtypes of disease that may look the same on the surface but respond very differently to treatment, tests that can identify disease carrier status for prospective parents, devices for managing diseases and for tracking and guiding recovery, or the like.

Examples of precision medicine may include receiving a user identifier identifying a user accessing a contextual interface to an electronic medical records (EMR) database. The EMR database may be accessed to obtain EMR data associated with the user identifier. In an example, a context attributed to the user accessing the contextual interface may be determined. A view of the EMR data may be presented within the contextual interface. In an example, the view is based on the user identifier or the context. The view may include treatment options tailored to a specific patient or adapted to the context of the user accessing the contextual interface.

The treatment options for the specific patient may include modifications based on genetic testing results. In an example, the treatment options for the specific patient include modifications based on a determined aggressiveness of a cancer diagnosis. In another example, the treatment options for the specific patient include further modifications based on a determined stage of the cancer diagnosis. The treatment options for the specific patient may include modifications based on an environmental factor associated with the patient.

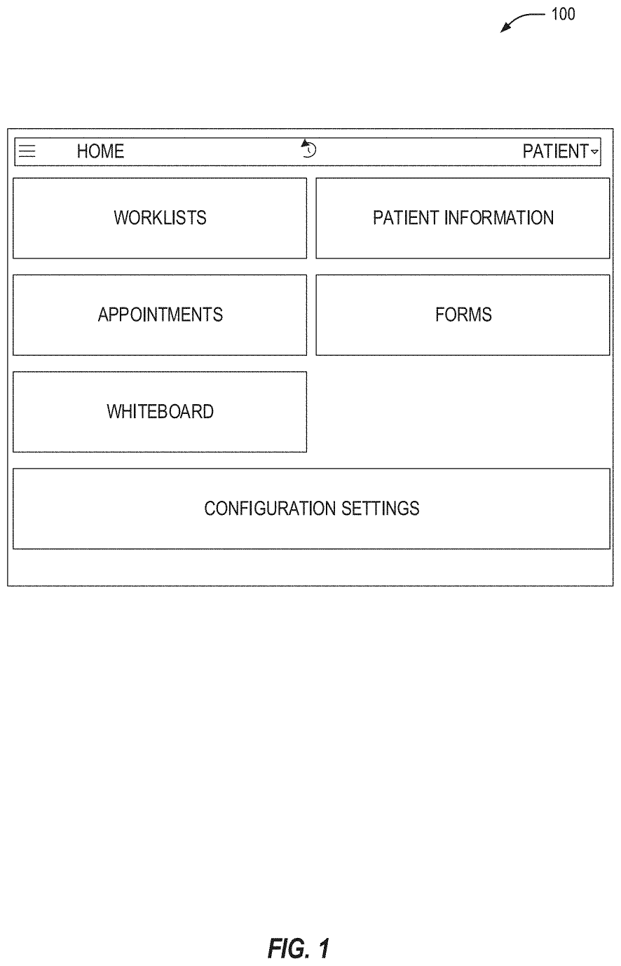

FIG. 1 illustrates an example user interface (UI) 100 for accessing different aspects of the systems and methods described herein, in accordance with at least one example of this disclosure. The UI 100 includes selectable options to view worklists, appointments, a whiteboard, patient details, forms, or configuration settings. In an example, the UI 100 is an interface of a program, for example an interface application layered on top of an EMR database system. One example interface application is SmartClinic, which may be a mobile or desktop interface application layered on top of an EMR database system, such as MOSAIQ® from Elekta Instrument AB of Stockholm, Sweden. SmartClinic provides a convenient interface for users to access Worklists, Appointments, Patient information, Dynamic Whiteboards, or Forms from a network connected computing device. SmartClinic operates on personal computers, tablets, or smartphones.

Worklists provide a user centric view of documents and tasks awaiting attention. Users can read and approve documents, which can automatically trigger follow up actions within the EMR system. Task lists can be filtered in various ways to tailor views to the individual user's needs, which can streamline task completion and prioritization. Task views within SmartClinic allow for a user to reply with notes or questions to the task requester (owner) when additional information or clarification is needed. The reply can trigger a notification to the requester that action on the task is needed.

Appointments provide another user-centric contextual view of a user's calendar. Appointments can be viewed in an Agenda-style or by day, week, or month. Appointments can be filtered by user, department, or other appointment classifications. A user can navigate directly to patient detail data from within an appointment to streamline the ability to fully understand an upcoming appointment. Patient Synopsis is discussed further below.

SmartClinic implements SmartForms for data entry tasks. SmartForms are dynamic and display only the data necessarily to complete a particular task or fill out a form. For example, if an in-take form requires information about smoking cessation, an initial question regarding whether the patient is a smoker can trigger whether the form displays and requires additional input on smoking cessation. In an example, a user is only presented with the information or input necessary to complete a particular form or task, streamlining data entry tasks.

SmartForms can be created for any EMR data entry requirements through a what-you-see-is-what-you-get (WYSIWYG) editor. The SmartForms editor includes drag-n-drop form components linked to EMR data fields allowing form creation with no programming or special skills. SmartForms can also be triggered through the dynamic Whiteboards, which are discussed in further detail below.

SmartClinic may be used to initialize a form editing interface, such as by generating a blank data entry form. An input (e.g., a user input) may be received to add a form field into the data entry form. The form field may be linked to a data field within an EMR system. The SmartClinic may generate an executable data entry form containing the data field linked to the EMR system, in an example, the executable data entry form may be displayed on a user interface (e.g., UI 100).

Linking the form field to the data field may include receiving input based on a user selection from a dropdown list. The UI 100 may receive input creating a plurality of data input form fields, with each data input form field linked to an EMR data field. For example, a first data input form field of the plurality of data input form fields may control display of a portion of the remaining plurality of data input form fields. In an example, a portion of the plurality of data input form fields include default values based on common patient parameters.

For any oncology patient there is a dearth of data associated with their treatment and care. Electronic Medical Records (EMR) store all this data. But it is cumbersome to input the data, and difficult to see the forest from the trees, such as what is relevant for a particular patient at a particular time given the disease state, type of cancer, and various treatments provided. The systems and methods described herein provide a filter of the vast EMR data to present what is important and relevant in an easily, graphical configuration.

Patient Synopsis is a software program that allows for unique presentation of EMR data stored in Mosaiq. As more information is loaded into Patient Synopsis, it becomes dynamic in its ability to provide the user what should be displayed. Various snipes are presented based on the context a user wants. For instance, a radiation oncologist will want something different from a dosimetrist or a nurse. Multiple views may be built and the system determines what is relevant to display.

Different data types may be detected when capturing data. This saves the user time and avoids needing require hundreds of mouse clicks to enter data. As the data is captured it is tailored. For example, there are tailored assessments based on gender.

In another example, Patient Synopsis may detect the context of the data. The Patient Synopsis takes into account the type of cancer, (e.g., is it breast cancer, lung cancer, or prostate cancer etc.), the co-morbidities, or the like. Further, the Patient Synopsis takes into account the patient's complications or the various procedures (e.g., chemotherapy) used.

An example method includes receiving a user identifier identifying a user accessing a dynamic electronic whiteboard. EMR data associated with the user identifier from the EMR database may be accessed. A context attributed to the user accessing the dynamic electronic whiteboard may be determined. The context may include a user's job or task list, such as based on a user ID or a user login. The context may include a clinic context, such as whether the patient is going to be at the clinic on a given day, what types of procedures may be performed (e.g., radiation or chemo), when a patient last had a procedure done, whether paperwork needs to be filled out (by the clinician or the patient), or the like. The context may include a patient context, such as co-morbidities, type of cancer, age, gender, medical history, etc. In an example, based on the context or the user identifier, a view of the EMR data may be presented. The view may filter or sort the EMR data displayed to customize the view to the user or the patient or both.

In an example, the method may include verifying the user is authorized to access the EMR database (e.g., based on login credentials, biometrics, etc.). The method may include determining the context includes determining a job classification associated with the user identifier. The job classification may include a radiation oncologist, a dosimetrist, a nurse, a physician's assistant, a clerk (e.g., a front desk clerk or other user interacting with a patient), or the like.

The user identifier may be used to determine a relevant patient (e.g., based on a time of day, what is the next patient for this clinician). Additional information for the relevant patient may be determined, such as a disease state, a type of cancer, past treatment information, or the like. This information may be used for the context or displayed within the user interface.

In an example the context includes tracing a navigation path associated with the user identifier, which may include triggering presentation of the view from a schedule. In an example, the view of the EMR data triggered from the schedule includes details associated with a selected appointment associated with the user identifier. In another example, tracing the navigation path includes triggering presentation of the view from a patient whiteboard. The view of the EMR data may include EMR data associated with a selected patient. The EMR data may be associated with at least one upcoming procedure scheduled for the selected patient.

In an example, the method may include receiving a user selection of a query for a patient evaluation operation. In this example, the user interface may present results of the query. Receiving the user selection may include receiving a methodology for extracting search results from the EMR database, for example by receiving one or more parameters for extracting the search results. The patient evaluation operation may include at least one of a retrospective patient medical chart review operation, a clinical trial matching operation, a quality assurance operation for a health care system, or a medical outcome prediction operation.

The EMR database may include unstructured natural language content (e.g., notes) or structured information content (e.g., demographic information for a patient, type of cancer, co-morbidities, treatment or procedure information, etc.). The EMR database may include detailed information for a particular patient, including procedures performed on the patient, interactions with medical practitioners concerning the patient, medications associated with the patient, results of medical tests and procedures, patient demographic information, static medical condition information, or the like.

Executing a query against the EMR database may include using one or more logical search engines to extract the search results. The search results in this example may include at least one matched natural language term.

The example method and techniques described above may be implemented using an EMR system including an EMR database and a dynamic electronic whiteboard hosted on a computing device displayed via a custom user interface (e.g., at a display screen of or coupled to the computing device). The computing device may include a processor and a memory device, the memory device including instructions that, when executed by the processor, cause the computing device to perform any of the methods or techniques described above.

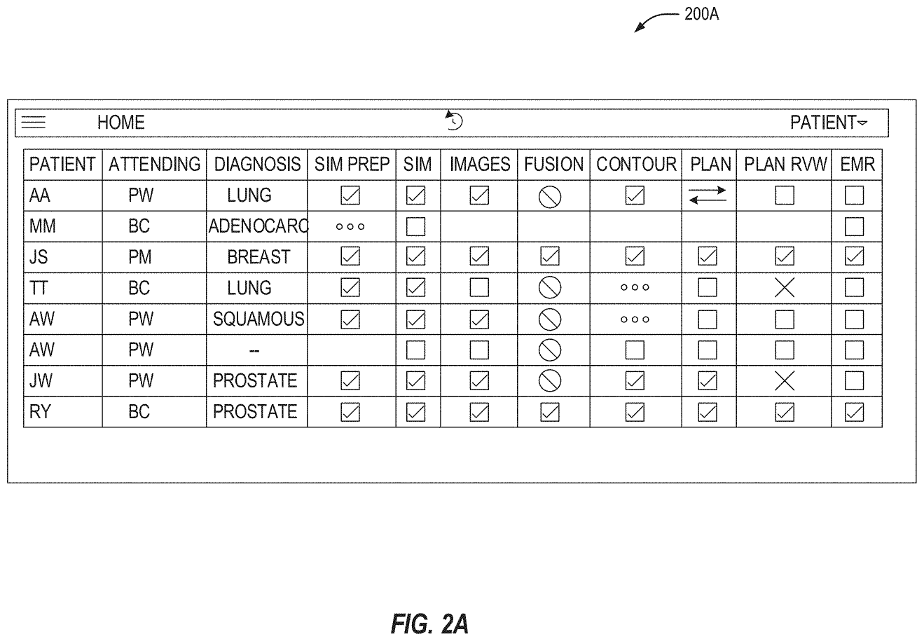

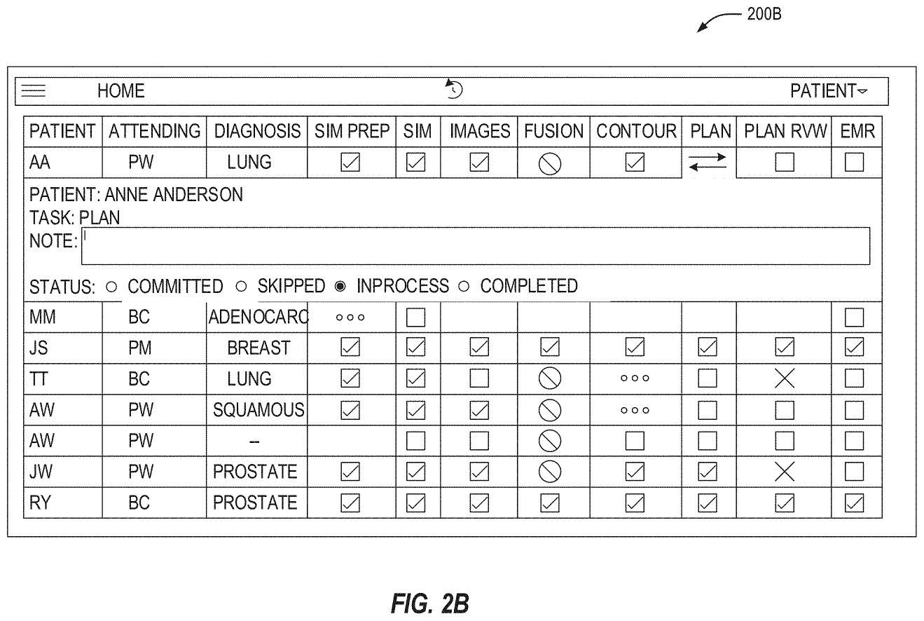

FIGS. 2A-2B illustrate a dynamic patient whiteboard in a custom user interface 200A and 200B, in accordance with at least one example of this disclosure.

For any oncology patient there is a dearth of data associated with their treatment and care. Electronic Medical Records (EMR) store all this data. But it is cumbersome to input the data, and difficult to see the forest from the trees, such as what is relevant for a particular patient at a particular time given the disease state, type of cancer, and various treatments provided. The systems and methods described herein provide a visualization of the tasks to be performed, automation as to whether a task is complete or ready to be completed, and notification to the user, even when the user is not using a particular program or app.

The system interfaces with electronic medical records (EMR) database software to acquire data from its database. In an example, the whiteboard (e.g., 200A or 200B) provides a visualization of the tasks to be performed, what tasks are ready to be performed, and what has been performed. For instance, during a patient assessment, the system can navigate a myriad of templates (e.g., SmartForms) or questions to provide a smart selection based on whether the patient is a new patient, a patient currently having treatment, a breast patient, a prostate patient, or a lung patient, among others. The system automatically can verify whether tasks are ready, or being performed without the user having to check. The whiteboard (e.g., 200A or 200B) verifies that multiple tasks are ready and whether tasks are being performed concurrently, unlike other systems that are linear. In an example, the system provides user notification. If a task is ready to go, a user is alerted. The whiteboard (e.g., 200A or 200B) is able to provide messages to the user's device even if the user is not actively viewing or using the whiteboard (e.g., 200A or 200B). A contextual workflow is tailored, in that, the right information is provided for a particular type of patient.

The system can generate multiple different whiteboards (e.g., 200A or 200B) for different contexts or user groups. For example, the system can host whiteboards focused on SIM-Ready to Treat, Consult Visits. Clinic Boards, On Treatment Boards, or Prescription Change Boards, which may all be configured to display different sets of data and tasks (e.g., support different workflows).

Whiteboards can be used to complete tasks and trigger automation that sets up follow up tasks or sends notifications, among other things. SmartForms can be launched directly from tasks shown on a whiteboard, and enable direct streamlined completion of certain tasks (e.g., plan review).

An example SIM-Ready to Treat whiteboard is shown in FIG. 2A. The whiteboard 200A includes columns for all tasks within the workflow for this whiteboard 20A. For each patient, the whiteboard 200A dynamically updates status of the tasks based on data within the EMR system. As the data within the EMR system is changed, the whiteboard 200A automatically updates the appropriate tasks. A check box indicates a task has been successfully completed. An X indicates the task was rejected. An ellipsis ( . . . ) indicates a task is ready to be performed, but not yet in process. Opposing arrows indicate a task is in process. Blank boxes indicate tasks that will need to be completed, but that are not yet ready. Blanks in columns indicate that the task is not necessary for this patient (or at least not currently necessary). For example, if a treat plan is reviewed successfully the RePlan task may not become relevant for that patient.

The whiteboard 200B of FIG. 2B illustrates an example of how a whiteboard can enable direct data input and task completion. Here, the Plan task for Anne Anderson has been selected and details shown. The details section allows for entry of notes and completion of the task.

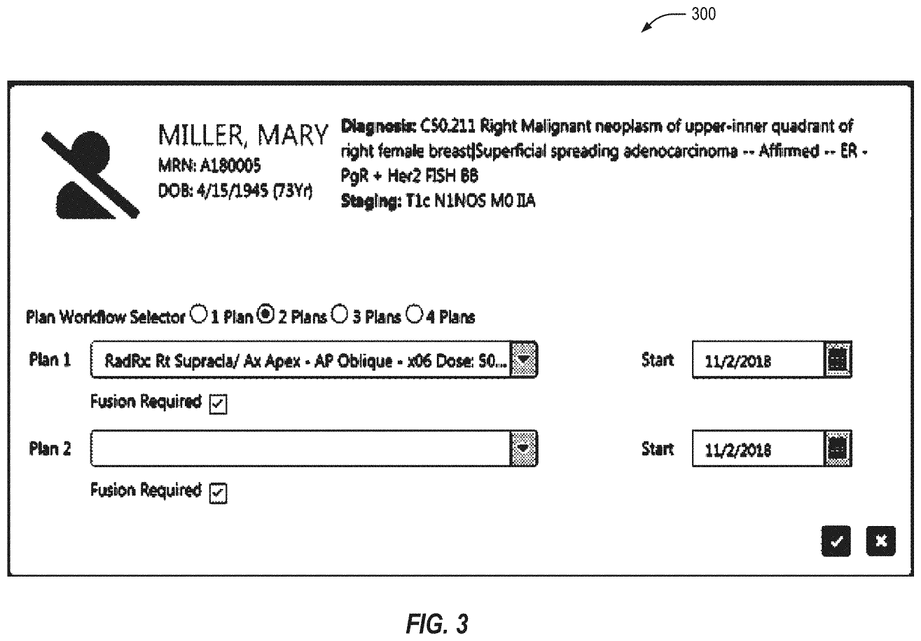

FIG. 3 illustrates a smart form in a custom user interface 300, in accordance with at least one example of this disclosure.

The next images illustrate how SmartForms can operate within the Whiteboard context to provide direct streamlined input. The SmartForm illustrates a form for providing treatment plan input. The custom user interface 300 illustrates how selection of an input within a form can dynamically change the SmartForm. In this example, the “2 Plans” option has been selected and the “Plan 2” text with supporting user interface components has been generated.

The changes to the SmartForm (e.g., 300) may dynamically change the whiteboard (e.g., 200A or 200B). In an example, adding a second treatment row for Mary Miller on the whiteboard may be based on the selection of 2 plans (from the SmartForm of FIG. 3).

A dynamic patient whiteboard may be displayed including a patient list including a plurality of patients, each patient of the plurality of patients in the patient list including a plurality of tasks, each task of the plurality of task including a task status. An EMR database may be accessed concurrently to displaying the dynamic patient whiteboard. In response to accessing the EMR database, it may be determined whether the task status for any of the plurality of concurrent tasks for each patient of the plurality of patients has updated. In an example, in response to an updated task status, the dynamic patient whiteboard may be updated to reflect the updated task status. Context information for each updated task may be displayed, for example based upon a particular user identity.

The task status for the dynamic patient whiteboard may include “ready to be completed,” “pending,” “in-process,” or “completed.” A notification may be sent to an owner of the task of the plurality of tasks with the task status. The task statuses may be color-coded, in an example. The dynamic patient whiteboard may include a color-coded patient list, such as according to disease state, type of cancer, treatment protocol, context (e.g., job classification), task status, or the like.

An input may be received on the dynamic patient whiteboard selecting a task of the plurality of tasks for a patient of the plurality of patients. In response to selection of the task, the dynamic patient whiteboard may display a template associated with task, and the template may include steps associated with completing the task. In an example, the dynamic patient whiteboard automatically inputs data from the content of dictation without user intervention. In another example, the dynamic patient whiteboard automatically sends an instant message to a user, which upon receipt the whiteboard includes into the EMR. In an example, dynamic patient whiteboard includes a plurality of preset commands, the preset commands may include a set of instructions when executed instruct the processor to access information and include into the EMR.

The dynamic patient whiteboard may be hosted on a computing device communicatively coupled to an electronic medical records (EMR) database. The computing device may include a processor and a memory device, the memory device including instructions that, when executed by the processor, cause the computing device to display the dynamic patient whiteboard.

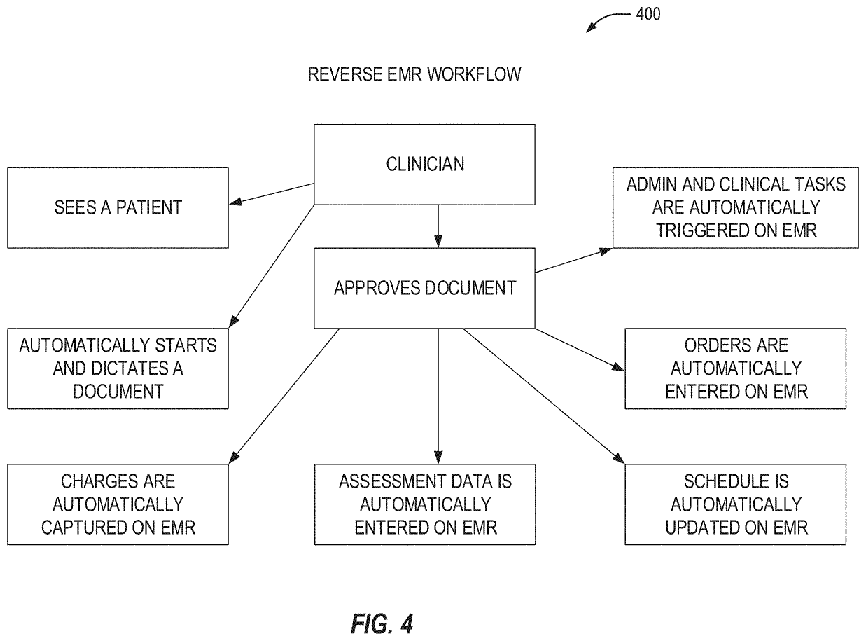

FIG. 4 illustrates a reverse electronic medical records (EMR) workflow 400, in accordance with at least one example of this disclosure.

Reverse EMR (REMR) changes the paradigm of patient documentation because it can automatically trigger clinically relevant actions in the Electronic Medical Record (EMR) based on content of the physician's dictation. With REMR, the document—which typically is just a byproduct of patient care—becomes an integral part of it, because when the document is approved, REMR automatically triggers orders, starts and completes tasks, captures charges, enters assessment data and changes schedule status, all based on the content of the dictation.

Traditional workflows include clinicians dictating a record of their patient visit on a dictaphone. A document is returned to them for review. Other example workflows use speech recognition technology to allow a clinician to create documentation inside the EMR and use speech recognition to dictate the information, utilizing some merge-fields that bring information from the chart. After dictating their notes, physicians still have to manually perform many tasks on the EMR. This is a click intensive/non-user friendly process that highly contributes to physician burnout. Documentation is still just a byproduct of the patient encounter.

Reverse EMR workflow is described herein. Reverse EMR technology makes the document a centerpiece of patient care. The clinician sees the patient and dictates the note. Clinical and admin activities and tasks—such as orders, charges, quality check lists, schedule status, etc. are all triggered automatically on document approval. The reverse EMR workflow allows for tasks and documentation to be automatically generated using dictation or smart forms.

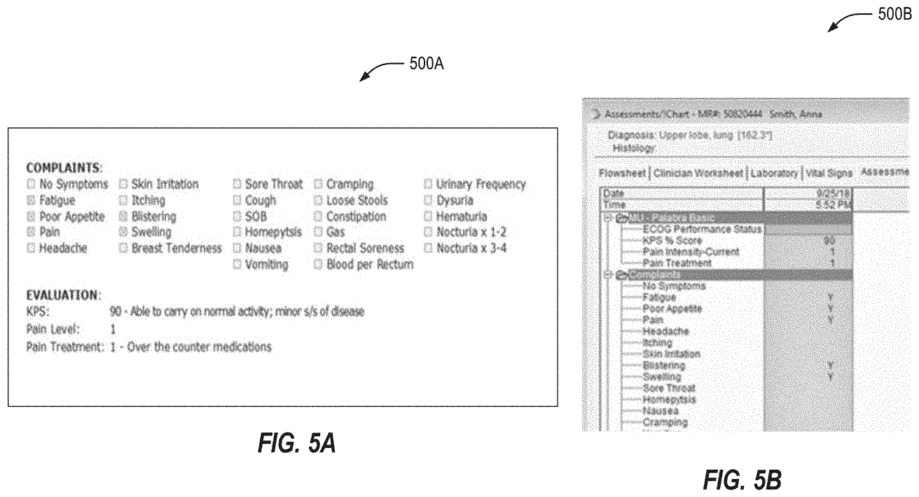

FIGS. 5A-5B, 6A-6H, 7A-7E, and 8A-8C illustrate example user interface components displaying aspects of a reverse EMR system, in accordance with at least one example of this disclosure.

FIGS. 5A-5B illustrate a reverse EMR Assessment Capture. EMRs offer users the option to enter patient assessment information. Often this information is used in the documentation to keep referring physicians updated of the patient progress. Typically, the information is entered in the EMR and then it has to be reproduced in the document. Reverse EMR allows the physician to enter the assessment data directly into the document, and then it automatically travels back to the EMR. A 2 step process becomes a 1 step process. FIG. 5A illustrates selectable options on a user interface 500A.

Specific fields (such as those shown on user interface 500A) in a document may be reverse EMR fields. Other fields may be separately displayed. The reverse EMR fields merge into the document when filled out in the EMR, and they may be sent back to the EMR when filled out in the document itself.

FIG. 5B illustrates a user interface with fields that were filled out in the document and automatically recorded on the EMR.

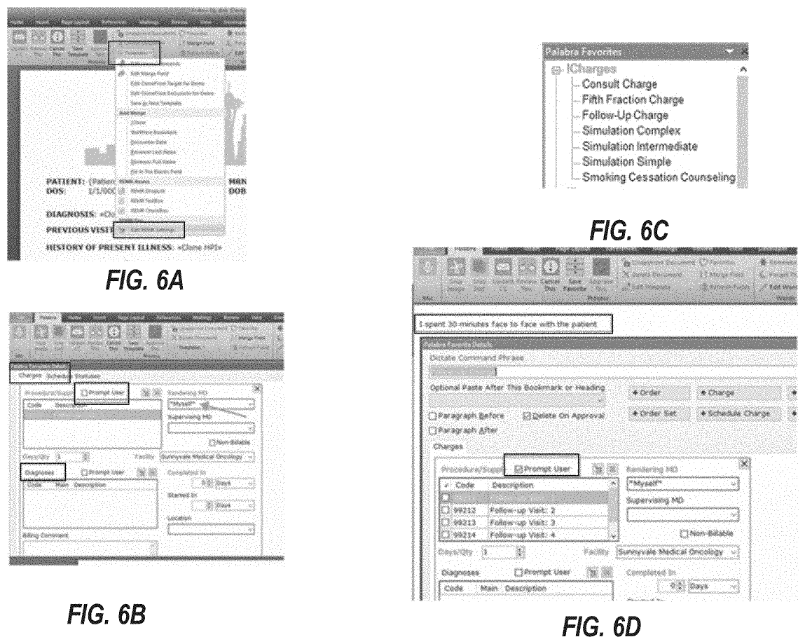

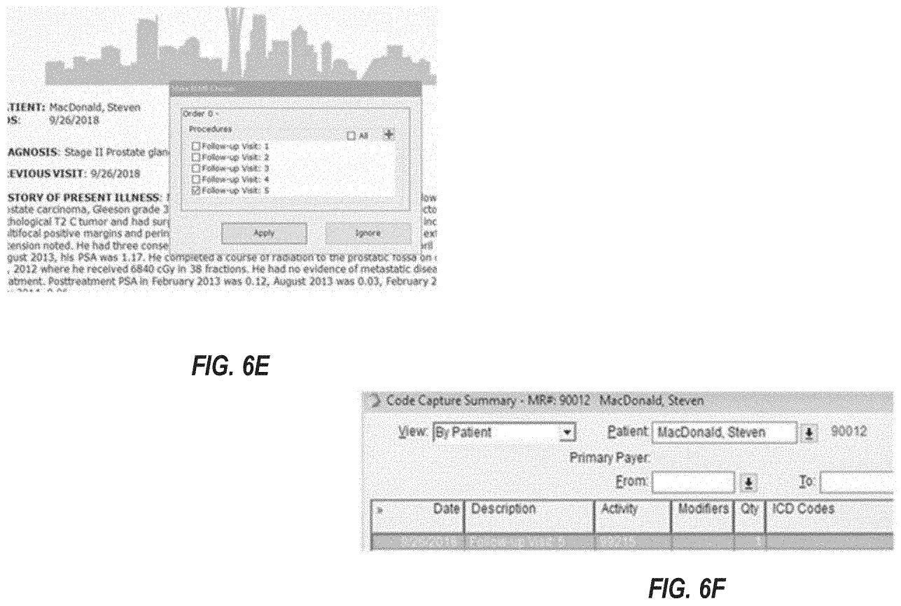

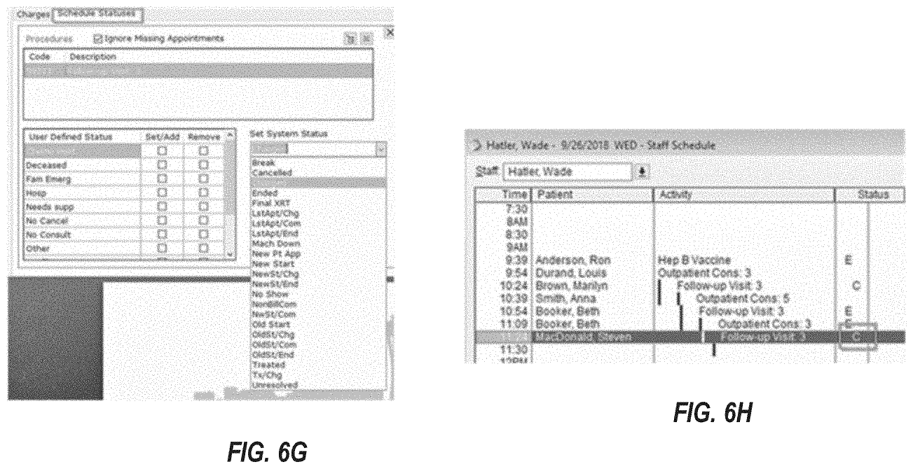

FIGS. 6A-6F illustrate a reverse EMR Charge Capture. Documentation plays a crucial part in charging for patient care. Every single charge has to be documented or it might be denied. Traditionally, billers have to read carefully the document to be able to charge accurately. With REMR, charges occur automatically either from the template (programmed to charge automatically), or from the content of the dictation.

The ribbon shown in FIG. 6A gives the user the option of editing the template REMR settings. When doing this, the user is programming the template to automatically execute certain actions on document approval.

FIG. 6B shows a charge that is attached to the template itself, such that, for example every time a note is approved, the change is automatically captured. The user can choose to be prompted. The charge can be attached to a particular diagnosis.

Changes can also be configured as reverse EMR favorites. Changes may be added to the dictation with a customizable voice command or a user input (e.g., finger touch or mouse click). These changes are shown for example in FIG. 6C.

FIG. 6D shows a favorite that configured to prompt the user to choose which Follow-Up charge to add to the document. Written justification for the charge can be added automatically to the document.

When the favorite is activated, the user chooses the pertinent charge, for example as shown on FIG. 6E.

FIG. 6F illustrates how, on approval of the document, the change is automatically entered on the EMR.

FIGS. 6G-6H illustrate a reverse EMR Schedule Status. Traditionally, the schedule has to be maintained manually. Every single change has to be manually entered, either by the physician or an admin. Reverse EMR can update the schedule automatically on approval of the note. The user can select different statuses as needed.

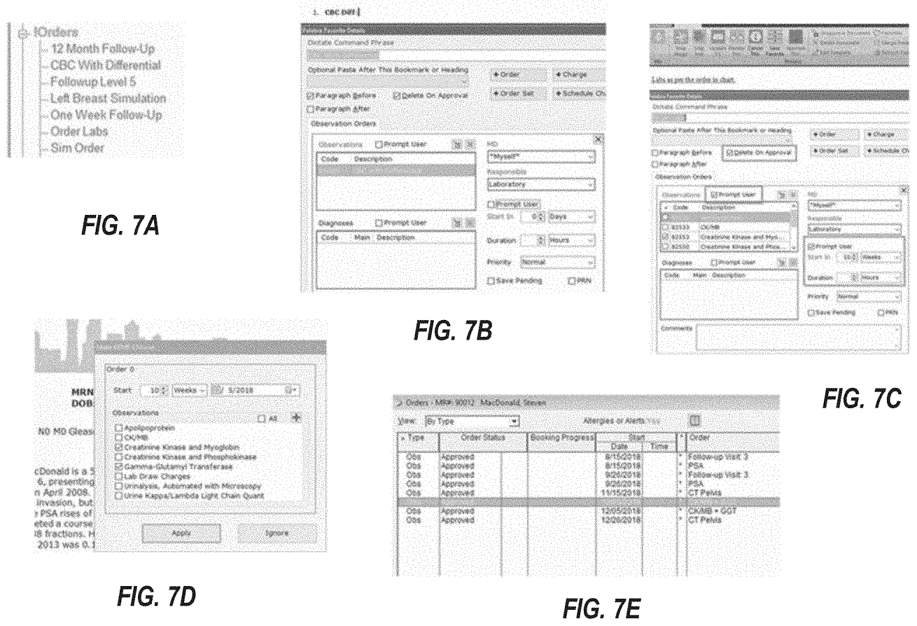

FIGS. 7A-7E illustrate automatic reverse EMR Orders. Orders are an important part of clinical care. Traditionally, doctors would verbally indicate to their staff whatever actions needed to be taken. Currently, EMRs allow to enter orders electronically, but this is a manual, click intensive process. Reverse EMR as shown in FIGS. 7A-7E allows a user to program orders on a very flexible manner and to enter orders with a voice command. FIG. 7A shows a list of orders. The user may enter or say a voice command “CBC with Differential” and everything else happens automatically. FIG. 7B shows a representation of how the order may be programmed.

The orders can be very complex and ask for resolution before being entered in the EMR. The user can be prompted to choose different options and can be prompted to decide when the order is due, for example as shown in FIG. 7D. There is an option for the clinician to click “apply” and take the default choices or make modifications as desired. When the document is approved, the orders are automatically entered on the EMR, as shown in FIG. 7E.

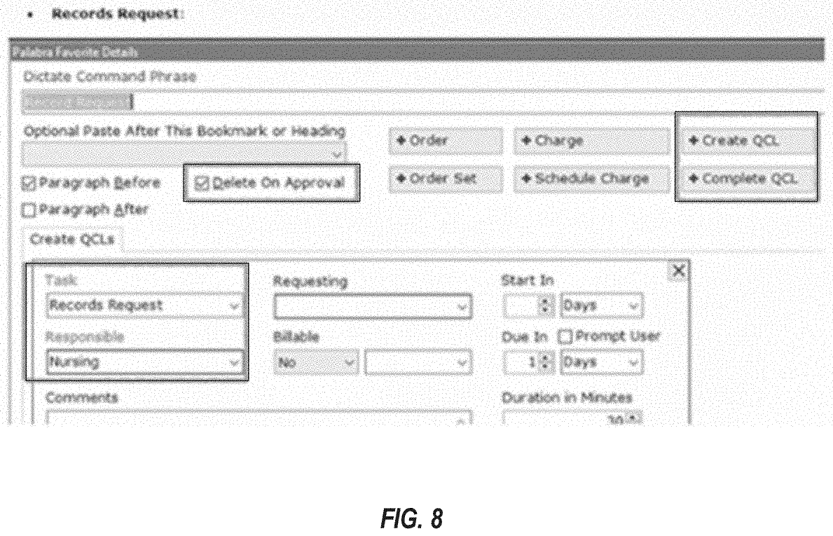

FIG. 8 illustrate reverse EMR Quality Check Lists (QCLs). QCLs are used to keep track of tasks that need to be completed. As with the other items, the REMR QCLs can be attached both to the template itself and be added ad-hoc as favorites. The mechanism is the same and they are also activated by voice commands. It is possible to create a QCL and also to complete a pending one. FIG. 8 includes an option to “delete on approval” which deletes a comment from the document (or all comments). QCLs may be used internally and the comments may be removed to create a clean document for outside use. The comments may appear in the EMR but not in the document, in an example.

When the physician finishes the document, and approves the note, actions that were triggered by a voice command or were programmed on the template (e.g., via default programming, or custom changes) gets executed. The physician may be presented with a summary of everything that is happening.

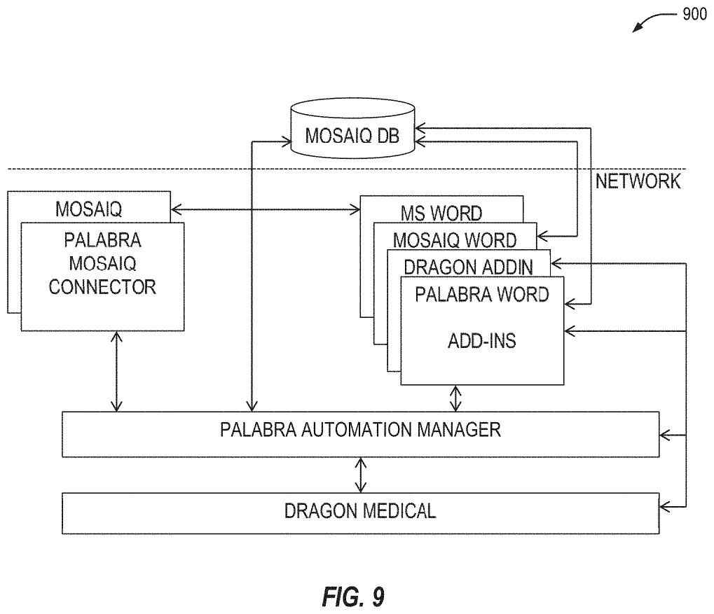

FIG. 9 illustrates a block diagram 900 of a system for presenting a dynamic patient whiteboard, in accordance with at least one example of this disclosure.

The simplified block diagram 900 shows a system architecture. Each of the groups represents a process boundary. All processes below the network line may be in a same window session, which may include the same workstation on local installations, and on the server for remote installations.

The systems and methods described herein may be fully integrated with MOSAIQ and the user interface may be entirely inside of the MOSAIQ application. A voice command can find a patient and start a document without a single mouse click in an example. From then on, navigation inside some MOSAIQ windows, and inside of the document can be executed by voice. The reverse EMR functions can be executed by voice. The user interfaces with forms when programming orders, charges, QCLs, etc.

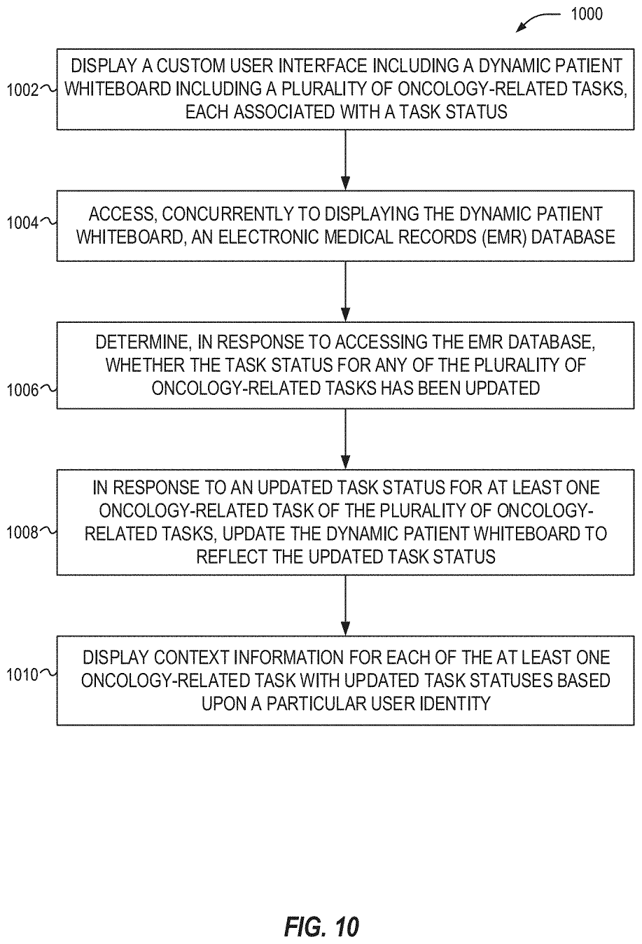

FIG. 10 illustrates a flowchart showing a technique 1000 for presenting a dynamic patient whiteboard in accordance with at least one example of this disclosure.

The technique 1000 includes an operation 1002 to display a custom user interface including a dynamic patient whiteboard including a plurality of oncology-related tasks, each associated with a task status. The dynamic patient whiteboard may include a patient list including a plurality of patients and related disease state, cancer type, or treatment protocol. In an example, each patient of the plurality of patients in the patient list may include a plurality of oncology-related tasks. An oncology related task may include a task status, such as “ready to be completed,” “pending,” “in-process,” “completed,” or the like. Operation 1002 may include presenting a view of EMR data corresponding to the patient list based on a user identifier identifying a user accessing the dynamic patient whiteboard. The user identifier may include a job classification, such as a radiation oncologist, a dosimetrist, a nurse, a physician's assistant, a clerk (e.g., a front desk clerk or other user interacting with a patient), or the like. This example may include verifying that the user is authorized to access the EMR database before presenting the view of the EMR data.

The technique 1000 includes an operation 1004 to access, concurrently to displaying the dynamic patient whiteboard, an EMR database. The technique 1000 includes an operation 1006 to determine, for example using a processor, in response to accessing the EMR database, whether the task status for any of the plurality of oncology-related tasks has been updated, such as for each patient of the plurality of patients. The technique 1000 includes an operation 1008 to in response to an updated task status for at least one oncology-related task of the plurality of oncology-related tasks, update the dynamic patient whiteboard to reflect the updated task status.

The technique 1000 includes an operation 1010 to display context information for each of the at least one oncology-related task with updated task statuses based upon a particular user identity. In an example, the context information is selected based on a customization corresponding to the particular user identity. Operation 1010 may include displaying whether similar tasks are performed across a patient database, for example for similar patients (e.g., based on co-morbidities, cancer type, demographic information, etc.). In an example, when two patients have a same disease state or type of cancer, operation 1010 may include displaying differences or similarities in protocols used for the two patients. Displaying the differences may include displaying a suggestion for a change to a protocol for a patient based on the differences. In an example, the context information may be based on an order of tasks of the plurality of oncology-related tasks, a responsible individual for tasks of the plurality of oncology-related tasks, a timeline for tasks of the plurality of oncology-related tasks to be completed, or the like.

In an example, displaying the dynamic patient whiteboard includes color-coding the patient list according to the related disease state, cancer type, or treatment protocol. The technique 1000 may include receiving an input selecting an oncology-related task of the plurality of oncology-related tasks for a patient of the plurality of patients, and in response to selecting the oncology-related task, displaying a template associated with the oncology-related task, the template including steps associated with completing the oncology-related task. The template may include user-specified forms or formats. The dynamic patient whiteboard may include a plurality of preset commands, the preset commands including for example, a set of instructions, which when executed, instruct the processor to access information for inclusion into the EMR database. In an example, a document template is created using a form editing interface and a received user input on the form editing interface to add an oncology-related form field, the oncology-related form field related to a data field within the EMR database, the document template used to display the context information.

In an example, the dynamic patient whiteboard is configured to automatically receive and display data from a dictation without user intervention. In a example, the dynamic patient whiteboard automatically sends an instant message to a user, which upon receipt, the dynamic patient whiteboard includes into the EMR database. In an example, the technique 1000 may be implemented on a desktop computer, a mobile device (e.g., a cell phone or a tablet), for example, displaying the dynamic patient whiteboard on a display screen of a mobile device.

As previously discussed, respective electronic computing systems or devices may implement one or more of the methods or functional operations as discussed herein. In various embodiments, such electronic computing systems or devices operates as a standalone device or may be connected (e.g., networked) to other machines. For instance, such computing systems or devices may operate in the capacity of a server or a client machine in server-client network environment, or as a peer machine in a peer-to-peer (or distributed) network environment. Features of computing systems or devices may be embodied by a personal computer (PC), a tablet PC, a Personal Digital Assistant (PDA), a cellular telephone, a web appliance, or any machine capable of executing instructions (sequential or otherwise) that specify actions to be taken by that machine.

As also indicated above, the functionality discussed above may be implemented by instructions, logic, or other information storage on a machine readable medium. While the machine-readable medium may have been described in various examples with reference to be a single medium, the term “machine-readable medium” may include a single medium or multiple media (e.g., a centralized or distributed database, and/or associated caches and servers) that store the one or more transitory or non-transitory instructions or data structures. The term “machine-readable medium” shall also be taken to include any tangible medium that is capable of storing, encoding or carrying transitory or non-transitory instructions for execution by the machine and that cause the machine to perform any one or more of the methodologies of the present invention, or that is capable of storing, encoding or carrying data structures utilized by or associated with such instructions.

The above description includes references to the accompanying exhibits, attachments, and drawings, which form a part of the description. The drawings show, by way of illustration but not by way of limitation, specific embodiments in which the invention can be practiced. These embodiments are also referred to herein as “examples.” Such examples can include elements in addition to those shown or described. However, this disclosure also contemplates examples in which only those elements shown or described are provided. Moreover, the disclosure also contemplates examples using any combination or permutation of those elements shown or described (or one or more aspects thereof), either with respect to a particular example (or one or more aspects thereof), or with respect to other examples (or one or more aspects thereof) shown or described herein.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a,” “an,” “the,” and “said” are used when introducing elements of aspects of the invention or in the embodiments thereof, as is common in patent documents, to include one or more than one or more of the elements, independent of any other instances or usages of “at least one” or “one or more.” In this document, the term “or” is used to refer to a nonexclusive or, such that “A or B” includes “A but not B,” “B but not A,” and “A and B,” unless otherwise indicated.

In the appended claims, the terms “including” and “in which” are used as the plain-English equivalents of the respective terms “comprising” and “wherein.” Also, in the following claims, the terms “comprising,” “including,” and “having” are intended to be open-ended to mean that there may be additional elements other than the listed elements, such that after such a term (e.g., comprising, including, having) in a claim are still deemed to fall within the scope of that claim. Moreover, in the following claims, the terms “first,” “second,” and “third,” etc., are used merely as labels, and are not intended to impose numerical requirements on their objects.

The present invention also relates to a computing system adapted, configured, or operated for performing the operations herein. This system may be specially constructed for the required purposes, or it may comprise a general purpose computer selectively activated or reconfigured by a computer program (e.g., instructions, code, etc.) stored in the computer. The order of execution or performance of the operations in embodiments of the invention illustrated and described herein is not essential, unless otherwise specified. That is, the operations may be performed in any order, unless otherwise specified, and embodiments of the invention may include additional or fewer operations than those disclosed herein. For example, it is contemplated that executing or performing a particular operation before, contemporaneously with, or after another operation is within the scope of aspects of the invention.

In view of the above, it will be seen that the several objects of the invention are achieved and other advantageous results attained. Having described aspects of the invention in detail, it will be apparent that modifications and variations are possible without departing from the scope of aspects of the invention as defined in the appended claims. As various changes could be made in the above constructions, products, and methods without departing from the scope of aspects of the invention, it is intended that all matter contained in the above description and shown in the accompanying drawings shall be interpreted as illustrative and not in a limiting sense.

The examples described herein may be implemented in a variety of embodiments. For example, one embodiment includes a computing device including processing hardware (e.g., a processor or other processing circuitry) and memory hardware (e.g., a storage device or volatile memory) including instructions embodied thereon, such that the instructions, which when executed by the processing hardware, cause the computing device to implement, perform, or coordinate the electronic operations for these techniques and system configurations. Another embodiment discussed herein includes a computer program product, such as may be embodied by a machine-readable medium or other storage device, which provides the transitory or non-transitory instructions to implement, perform, or coordinate the electronic operations for these techniques and system configurations. Another embodiment discussed herein includes a method operable on processing hardware of the computing device, to implement, perform, or coordinate the electronic operations for these techniques and system configurations.

In further embodiments, the logic, commands, or transitory or non-transitory instructions that implement aspects of the electronic operations described above, may be provided in a distributed or centralized computing system, including any number of form factors for the computing system such as desktop or notebook personal computers, mobile devices such as tablets, netbooks, and smartphones, client terminals and server-hosted machine instances, and the like. Another embodiment discussed herein includes the incorporation of the techniques discussed herein into other forms, including into other forms of programmed logic, hardware configurations, or specialized components or modules, including an apparatus with respective means to perform the functions of such techniques. The respective algorithms used to implement the functions of such techniques may include a sequence of some or all of the electronic operations described above, or other aspects depicted in the accompanying drawings and detailed description below.

The above description is intended to be illustrative, and not restrictive. For example, the above-described examples (or one or more aspects thereof) may be used in combination with each other. In addition, many modifications may be made to adapt a particular situation or material to the teachings of the invention without departing from its scope. While the dimensions, types of materials and example parameters, functions, and implementations described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled.

Also, in the above description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

Example 1 is a method for presenting a dynamic patient whiteboard, the method comprising: displaying a custom user interface including the dynamic patient whiteboard comprising a patient list including a plurality of patients and related disease state, cancer type, or treatment protocol, each patient of the plurality of patients in the patient list including a plurality of oncology-related tasks, each oncology-related task of the plurality of oncology-related tasks including a task status; accessing, concurrently to displaying the dynamic patient whiteboard, an electronic medical records (EMR) database; determining, using a processor, in response to accessing the EMR database, whether the task status for any of the plurality of oncology-related tasks for each patient of the plurality of patients has been updated; in response to an updated task status for at least one oncology-related task of the plurality of oncology-related tasks, updating the dynamic patient whiteboard to reflect the updated task status; and displaying context information for each of the at least one oncology-related task with updated task statuses based upon a particular user identity, the context information selected based on a customization corresponding to the particular user identity.

In Example 2, the subject matter of Example 1 includes, wherein displaying the dynamic patient whiteboard includes displaying task statuses of corresponding oncology-related tasks including “ready to be completed”. “pending”, “in-process”, and “completed”.

In Example 3, the subject matter of Examples 1-2 includes, wherein displaying the dynamic patient whiteboard includes color-coding the patient list according to the related disease state, cancer type, or treatment protocol.

In Example 4, the subject matter of Examples 1-3 includes, receiving an input selecting an oncology-related task of the plurality of oncology-related tasks for a patient of the plurality of patients, and in response to selecting the oncology-related task, displaying a template associated with the oncology-related task, the template including steps associated with completing the oncology-related task.

In Example 5, the subject matter of Examples 1-4 includes, wherein the dynamic patient whiteboard is configured to automatically receive and display data from a dictation without user intervention.

In Example 6, the subject matter of Examples 1-5 includes, wherein the dynamic patient whiteboard automatically sends an instant message to a user, which upon receipt, the dynamic patient whiteboard includes into the EMR database.

In Example 7, the subject matter of Examples 1-6 includes, wherein the dynamic patient whiteboard includes a plurality of preset commands, the preset commands including a set of instructions, which when executed, instruct the processor to access information for inclusion into the EMR database.

In Example 8, the subject matter of Examples 1-7 includes, wherein displaying context information includes displaying whether similar tasks are performed across a patient database.

In Example 9, the subject matter of Examples 1-8 includes, wherein when two patients have a same disease state or type of cancer, displaying the context information includes displaying differences in protocols used for the two patients.

In Example 10, the subject matter of Example 9 includes, wherein displaying the context information includes displaying a suggestion for a change to a protocol for a patient based on the differences.

In Example 11, the subject matter of Examples 1-10 includes, wherein displaying the dynamic patient whiteboard includes presenting a view of EMR data corresponding to the patient list based on a user identifier identifying a user accessing the dynamic patient whiteboard.

In Example 12, the subject matter of Example 11 includes, verifying the user is authorized to access the EMR database before presenting the view of the EMR data.

In Example 13, the subject matter of Examples 11-12 includes, wherein the user identifier includes a job classification.

In Example 14, the subject matter of Examples 1-13 includes, wherein displaying the dynamic patient whiteboard includes displaying the dynamic patient whiteboard on a display screen of a mobile device.

In Example 15, the subject matter of Examples 1-14 includes, wherein the context information is based on an order of tasks of the plurality of oncology-related tasks, a responsible individual for tasks of the plurality of oncology-related tasks, and a timeline for tasks of the plurality of oncology-related tasks.

In Example 16, the subject matter of Examples 1-15 includes, wherein a document template is created using a form editing interface and a received user input on the form editing interface to add an oncology-related form field, the oncology-related form field related to a data field within the EMR database, the document template used to display the context information.

Example 17 is a computing device for hosting a dynamic patient whiteboard, the computing device communicatively coupled to an electronic medical records (EMR) database, the computing device including a processor and a memory device, the memory device including instructions that, when executed by the processor, cause the computing device to: display a custom user interface including the dynamic patient whiteboard comprising a patient list including a plurality of patients and related disease state, cancer type, or treatment protocol, each patient of the plurality of patients in the patient list including a plurality of oncology-related tasks, each oncology-related task of the plurality of oncology-related tasks including a task status; access, concurrently to displaying the dynamic patient whiteboard, the EMR database; determine, in response to accessing the EMR database, whether the task status for any of the plurality of oncology-related tasks for each patient of the plurality of patients has been updated; in response to an updated task status for at least one oncology-related task of the plurality of oncology-related tasks, update the dynamic patient whiteboard to reflect the updated task status; and display context information for each of the at least one oncology-related tasks with updated task statuses based upon a particular user identity, the context information selected based on a customization corresponding to the particular user identity.

In Example 18, the subject matter of Example 17 includes, wherein the dynamic patient whiteboard is displayed with color-coding of the patient list according to the related disease state, cancer type, or treatment protocol.

In Example 19, the subject matter of Examples 17-18 includes, wherein the computing device is a mobile device and the dynamic patient whiteboard is displayed on a display screen of the mobile device.

In Example 20, the subject matter of Examples 17-19 includes, wherein the dynamic patient whiteboard automatically sends an instant message to a user, which upon receipt, the dynamic patient whiteboard includes into the EMR database.

In Example 21, the subject matter of Examples 17-20 includes, wherein the dynamic patient whiteboard includes a plurality of preset commands, the preset commands including a set of instructions, which when executed, instruct the processor to access information for inclusion into the EMR database.

Example 22 is at least one machine-readable medium including instructions for hosting a dynamic patient whiteboard, which when executed by a processor, cause the processor to: display a custom user interface including the dynamic patient whiteboard comprising a patient list including a plurality of patients and related disease state, cancer type, or treatment protocol, each patient of the plurality of patients in the patient list including a plurality of oncology-related tasks, each oncology-related task of the plurality of oncology-related tasks including a task status; access, concurrently to displaying the dynamic patient whiteboard, an electronic medical records (EMR) database; determine, in response to accessing the EMR database, whether the task status for any of the plurality of oncology-related tasks for each patient of the plurality of patients has been updated; in response to an updated task status for at least one oncology-related task of the plurality of oncology-related tasks, update the dynamic patient whiteboard to reflect the updated task status; and display context information for each of the at least one oncology-related tasks with updated task statuses based upon a particular user identity, the context information selected based on a customization corresponding to the particular user identity.

In Example 23, the subject matter of Example 22 includes, wherein to display the dynamic patient whiteboard, the processor is further caused to display task statuses of corresponding oncology-related tasks including “ready to be completed”, “pending”, “in-process”, and “completed”.

In Example 24, the subject matter of Examples 22-23 includes, wherein to display the dynamic patient whiteboard, the processor is further caused to display the patient list color-coded according to the related disease state, cancer type, or treatment protocol.

Example 25 is a method for capturing clinical data and triggering follow-up activities, the method comprising: receiving dictation from a physician, the dictation including details from an interaction with an oncology patient; automatically, in response to receiving the dictation, generating text corresponding to the dictation; extracting electronic medical record (EMR) data from the text; generating a document including the EMR data and additional patient-specific EMR data for the oncology patient accessed via an EMR database; presenting the document in a user interface for approval by the physician; and triggering, automatically in response to receiving the approval, a series of EMR-related actions for the oncology patient based at least in part on contents of the document, the series of EMR-related actions including recording the EMR data into the EMR database.

In Example 26, the subject matter of Example 25 includes, wherein the series of EMR-related actions include at least one of recording changes associated with the oncology patient interaction, parsing and recording assessment data from the oncology patient interaction, or updating a schedule associated either the oncology patient or the physician in reference to the oncology patient interaction.

In Example 27, the subject matter of Examples 25-26 includes, wherein the series of EMR-related actions include at least one of entering an order associated with the oncology patient interaction, triggering administrative tasks or clinical tasks associated with the oncology patient interaction, or updating a task status in response to an aspect of the oncology patient interaction.

In Example 28, the subject matter of Examples 25-27 includes, wherein generating the document includes selecting a document template from a plurality of available templates.

In Example 29, the subject matter of Example 28 includes, wherein generating the document includes appending preprogrammed actions to the document based on the selected document template, the preprogrammed actions specific to the selected document template.

In Example 30, the subject matter of Examples 28-29 includes, wherein the document template is created using a form editing interface and a received user input on the form editing interface to add an oncology-related form field, the oncology-related form field related to a data field within the EMR database.

In Example 31, the subject matter of Example 30 includes, wherein the oncology-related form field is linked to the data field based on a user selection from a dropdown list.

In Example 32, the subject matter of Examples 25-31 includes, parsing the text to determine the series of EMR-related actions.

Example 33 is a computing device communicatively coupled to an electronic medical records (EMR) database, the computing device including a processor and a memory device, the memory device including instructions that, when executed by the processor, cause the computing device to: receive dictation from a physician, the dictation including details from an interaction with an oncology patient; automatically, in response to receiving the dictation, generate text corresponding to the dictation; extract EMR data from the text; generate a document including the EMR data and additional patient-specific EMR data for the oncology patient accessed via the EMR database; present the document in a user interface for approval by the physician; and trigger, automatically in response to receiving the approval, a series of EMR-related actions for the oncology patient based at least in part on contents of the document, the series of EMR-related actions including recording the EMR data into the EMR database.

In Example 34, the subject matter of Example 33 includes, wherein the series of EMR-related actions include at least one of recording changes associated with the oncology patient interaction, parsing and recording assessment data from the oncology patient interaction, or updating a schedule associated either the oncology patient or the physician in reference to the patient interaction.

In Example 35, the subject matter of Examples 33-34 includes, wherein the series of EMR-related actions include at least one of entering an order associated with the oncology patient interaction, triggering administrative tasks or clinical tasks associated with the oncology patient interaction, or updating a task status in response to an aspect of the oncology patient interaction.

In Example 36, the subject matter of Examples 33-35 includes, wherein to generate the document, the computing device is further caused to select a document template from a plurality of available templates.

In Example 37, the subject matter of Example 36 includes, wherein to generate the document, the computing device is further caused to append preprogrammed actions to the document based on the selected document template the preprogrammed actions specific to the selected document template.

In Example 38, the subject matter of Examples 36-37 includes, wherein the document template is created using a form editing interface and a received user input on the form editing interface to add an oncology-related form field, the oncology-related form field related to a data field within the EMR database.

In Example 39, the subject matter of Example 38 includes, wherein the oncology-related form field is linked to the data field based on a user selection from a dropdown list.

In Example 40, the subject matter of Examples 33-39 includes, wherein the computing device is further caused to parse the text to determine the series of EMR-related actions.

Example 41 is at least one machine-readable medium including instructions, which when executed by a processor, cause the processor to: receive dictation from a physician, the dictation including details from an interaction with an oncology patient; automatically, in response to receiving the dictation, generate text corresponding to the dictation; extract electronic medical record (EMR) data from the text; generate a document including the EMR data and additional patient-specific EMR data for the oncology patient accessed via an EMR database; present the document in a user interface for approval by the physician; and trigger, automatically in response to receiving the approval, a series of EMR-related actions for the oncology patient based at least in part on contents of the document, the series of EMR-related actions including recording the EMR data into the EMR database.

In Example 42, the subject matter of Example 41 includes, wherein the series of EMR-related actions include at least one of recording changes associated with the oncology patient interaction, parsing and recording assessment data from the oncology patient interaction, or updating a schedule associated either the oncology patient or the physician in reference to the oncology patient interaction.

In Example 43, the subject matter of Examples 41-42 includes, wherein the series of EMR-related actions include at least one of entering an order associated with the oncology patient interaction, triggering administrative tasks or clinical tasks associated with the oncology patient interaction, or updating a task status in response to an aspect of the oncology patient interaction.

In Example 44, the subject matter of Examples 41-43 includes, wherein to generate the document includes instructions to select a document template from a plurality of available templates.

In Example 45, the subject matter of Example 44 includes, wherein to generate the document includes instructions to append preprogrammed actions to the document based on the selected document template, the preprogrammed actions specific to the selected document template.

In Example 46, the subject matter of Examples 44-45 includes, wherein the document template is created using a form editing interface and a received user input on the form editing interface to add an oncology-related form field, the oncology-related form field related to a data field within the EMR database.

In Example 47, the subject matter of Example 46 includes, wherein the oncology-related form field is linked to the data field based on a user selection from a dropdown list.

In Example 48, the subject matter of Examples 41-47 includes, wherein the instructions further cause the processor to parse the text to determine the series of EMR-related actions.

Example 49 is at least one machine-readable medium including instructions that, when executed by processing circuitry, cause the processing circuitry to perform operations to implement of any of Examples 1-48.

Example 50 is an apparatus comprising means to implement of any of Examples 1-48.

Example 51 is a system to implement of any of Examples 1-48.

Example 52 is a method to implement of any of Examples 1-48.

What is claimed is:

1. A method for presenting a dynamic patient whiteboard, the method comprising:
displaying a custom user interface including the dynamic patient whiteboard comprising a patient list including a plurality of patients and related disease state, cancer type, or treatment protocol, each patient of the plurality of patients in the patient list including a plurality of oncology-related tasks, each oncology-related task of the plurality of oncology-related tasks including a task status;
accessing, concurrently to displaying the dynamic patient whiteboard, an electronic medical records (EMR) database;
determining, using a processor, in response to accessing the EMR database, whether the task status for any of the plurality of oncology-related tasks for each patient of the plurality of patients has been updated;
in response to an updated task status for at least one oncology-related task of the plurality of oncology-related tasks, updating the dynamic patient whiteboard to reflect the updated task status; and
displaying context information for each of the at least one oncology-related task with updated task statuses based upon a particular user identity, the context information selected based on a customization corresponding to the particular user identity.

2. The method of claim 1, wherein displaying the dynamic patient whiteboard includes displaying task statuses of corresponding oncology-related tasks including “ready to be completed”, “pending”, “in-process”, and “completed”.

3. The method of claim 1, wherein displaying the dynamic patient whiteboard includes color-coding the patient list according to the related disease state, cancer type, or treatment protocol.

4. The method of claim 1, further comprising receiving an input selecting an oncology-related task of the plurality of oncology-related tasks for a patient of the plurality of patients, and in response to selecting the oncology-related task, displaying a template associated with the oncology-related task, the template including steps associated with completing the oncology-related task.

5. The method of claim 1, wherein the dynamic patient whiteboard is configured to automatically receive and display data from a dictation without user intervention.

6. The method of claim 1, wherein the dynamic patient whiteboard automatically sends an instant message to a user, which upon receipt, the dynamic patient whiteboard includes into the EMR database.

7. The method of claim 1, wherein the dynamic patient whiteboard includes a plurality of preset commands, the preset commands including a set of instructions, which when executed, instruct the processor to access information for inclusion into the EMR database.

8. The method of claim 1, wherein displaying context information includes displaying whether similar tasks are performed across a patient database.

9. The method of claim 1, wherein when two patients have a same disease state or type of cancer, displaying the context information includes displaying differences in protocols used for the two patients.

10. The method of claim 9, wherein displaying the context information includes displaying a suggestion for a change to a protocol for a patient based on the differences.

11. The method of claim 1, wherein displaying the dynamic patient whiteboard includes presenting a view of EMR data corresponding to the patient list based on a user identifier identifying a user accessing the dynamic patient whiteboard.

12. The method of claim 11, further comprising verifying the user is authorized to access the EMR database before presenting the view of the EMR data.

13. The method of claim 11, wherein the user identifier includes a job classification.

14. The method of claim 1, wherein displaying the dynamic patient whiteboard includes displaying the dynamic patient whiteboard on a display screen of a mobile device.

15. The method of claim 1, wherein the context information is based on an order of tasks of the plurality of oncology-related tasks, a responsible individual for tasks of the plurality of oncology-related tasks, and a timeline for tasks of the plurality of oncology-related tasks.

16. The method of claim 1, wherein a document template is created using a form editing interface and a received user input on the form editing interface to add an oncology-related form field, the oncology-related form field related to a data field within the EMR database, the document template used to display the context information.

17. A computing device for hosting a dynamic patient whiteboard, the computing device communicatively coupled to an electronic medical records (EMR) database, the computing device including a processor and a memory device, the memory device including instructions that, when executed by the processor, cause the computing device to:
display a custom user interface including the dynamic patient whiteboard comprising a patient list including a plurality of patients and related disease state, cancer type, or treatment protocol, each patient of the plurality of patients in the patient list including a plurality of oncology-related tasks, each oncology-related task of the plurality of oncology-related tasks including a task status;
access, concurrently to displaying the dynamic patient whiteboard, the EMR database;
determine, in response to accessing the EMR database, whether the task status for any of the plurality of oncology-related tasks for each patient of the plurality of patients has been updated;
in response to an updated task status for at least one oncology-related task of the plurality of oncology-related tasks, update the dynamic patient whiteboard to reflect the updated task status; and
display context information for each of the at least one oncology-related tasks with updated task statuses based upon a particular user identity, the context information selected based on a customization corresponding to the particular user identity.

18. The computing device of claim 17, wherein the dynamic patient whiteboard is displayed with color-coding of the patient list according to the related disease state, cancer type, or treatment protocol.

19. The computing device of claim 17, wherein the computing device is a mobile device and the dynamic patient whiteboard is displayed on a display screen of the mobile device.

20. The computing device of claim 17, wherein the dynamic patient whiteboard automatically sends an instant message to a user, which upon receipt, the dynamic patient whiteboard includes into the EMR database.

21. The computing device of claim 17, wherein the dynamic patient whiteboard includes a plurality of preset commands, the preset commands including a set of instructions, which when executed, instruct the processor to access information for inclusion into the EMR database.

22. At least one machine-readable medium including instructions for hosting a dynamic patient whiteboard, which when executed by a processor, cause the processor to:
display a custom user interface including the dynamic patient whiteboard comprising a patient list including a plurality of patients and related disease state, cancer type, or treatment protocol, each patient of the plurality of patients in the patient list including a plurality of oncology-related tasks, each oncology-related task of the plurality of oncology-related tasks including a task status;
access, concurrently to displaying the dynamic patient whiteboard, an electronic medical records (EMR) database;
determine, in response to accessing the EMR database, whether the task status for any of the plurality of oncology-related tasks for each patient of the plurality of patients has been updated;
in response to an updated task status for at least one oncology-related task of the plurality of oncology-related tasks, update the dynamic patient whiteboard to reflect the updated task status; and
display context information for each of the at least one oncology-related tasks with updated task statuses based upon a particular user identity, the context information selected based on a customization corresponding to the particular user identity.

23. The at least one machine-readable medium of claim 22, wherein to display the dynamic patient whiteboard, the processor is further caused to display task statuses of corresponding oncology-related tasks including “ready to be completed”, “pending”, “in-process”, and “completed”.

24. The at least one machine-readable medium of claim 22, wherein to display the dynamic patient whiteboard, the processor is further caused to display the patient list color-coded according to the related disease state, cancer type, or treatment protocol.
